(19) United States
(12) Patent Application Publication
(10) Pub. No.: US 2013/0172796 A1
(43) Pub. Date: Jul. 4, 2013

(54) CAST REMOVAL KIT AND METHOD

(71) Applicant: Olarewaju Oladipo, Canton, MA (US)

(72) Inventor: Olarewaju Oladipo, Canton, MA (US)

(21) Appl. No.: 13/342,054

(22) Filed: Jan. 1, 2012

(51) Int. Cl. A61F 5/01

(52) U.S. Cl. 602/9

(57) ABSTRACT

A device, kit and method thereof for removing an orthopedic cast.

### FIELD OF INVENTION

The present invention relates to the field of orthopedics, specifically treatment of fractures using casting materials. The invention of the cast removal kit is particularly relevant for use in emergency situations, in children, and patients who may be fearful of the use of a traditional cast saw. The invention of the cast removal kit is also ideal in circumstances where there is no electricity or a battery powered source is lacking to operate a traditional cast saw.

### BACKGROUND OF THE INVENTION

Treatment of bone fractures frequently requires the use of a cast. Removal of a cast is necessary at different stages of fracture healing. The traditional methods require the use of a shear or an electric saw.

The use of a shear is common in developing countries and in warfare setting where distance to optimal care is an issue. Its use however, is less than ideal because the lower cutting blade edge has to be pushed against the skin to initiate cutting. This maneuver exerts unnecessary pressure and discomfort on the skin and cases pain exacerbation when it is close to the fracture site.

The use of a cast saw is more efficient, but limited to settings where electricity or battery power packs are not readily available. The traditional cast saw also has the unusual effect of generating fear in younger children, or even adults who find the use of a saw unfriendly.

The use of the cast removal kit of this invention is a manual process requiring minimal effort, using the two handles attached to the two looped ends of the abrasive cord, sliding it back and forth with both hands in its protective sleeve until the cast material is fully cut.

### BRIEF DESCRIPTION OF THE PRESENT INVENTION

The present invention relates to a cast removal kit that is applicable when existing methods of cast removal is impractical due to the treating environment, or the patient's requirement.

The cast removal kit of the present invention consists of an elongated abrasive cord, having two attached loop—one at each end, preferably of non-abrasive material. The elongated abrasive cord is enclosed in a protective sleeve in the segment between its attached looped ends. The protective sleeve encases the elongated abrasive cord in a non-tight fitting manner, allowing for a little play within its lumen, such that the cord can be moved freely within it. The elongated protective sleeve is slit along its longitudinal axis such that the encased cord can be pulled through this opening during use. The elongated protective sleeve is of a flexible and collapsible material such that it will flatten when incorporated in a cast, and not exert unnecessary pressure on the underlying skin. The elongated protective sleeve is positioned during use with the slit end pointing upward such that the abrasive cord can be pulled through during use to cut on a casting material lying on top. The elongated protective sleeve is also of a material that can be cut through with the abrasive cord in the event that the longitudinal slit on the protective sleeve is malpositioned. The abrasive cord is made of one, or more strands of wire, or non-metallic material with similar cutting properties, the type that is already in use in the stone cutting industry and during bone cutting during hip replacement surgery.

The cast removal kit of the present invention consists of an abrasive cord partially enclosed in a protective sleeve for use in the removal of a cast on an extremity. During use, the abrasive cord with its protective sleeve is positioned deep to an applied cast, between the layer of protective padding that abuts the skin and the overlying casting material. The elongated protective sleeve with its enclosed abrasive cord is positioned during use with the slit end pointing upward or to the sides such that the abrasive cord can be pulled through during use to cut the overlying casting material. When applying a cast, at least one unit of the cast removal kit is positioned on either side of an applied extremity cast, buried deeper to the deepest layer of casting material, but above the layer of the layer of cast under-padding on the skin. To remove an applied cast, a pair of handles adapted to be easily gripped with the hands is fitted to the looped ends of the cord. Using a to and fro motion, tension is applied to the abrasive cord making it exit the slit opening along the length of the protective sleeve, and cutting through the overlying casting material. This process of cast removal is repeated on the two sides of the applied cast, using the pre-positioned cast removal kit. In the long extremity of an adult, it may be necessary to remove a small segment of an applied cast at a time, rather the whole length. As a result, more than one unit of the cast removal kit may be required per applied cast, with the kit positioned in series, or end-to-end along the length of a cast, and on two or more sides of an applied cast.

The cast removal kit of this invention consists of an abrasive cord with a loop at both ends, a protective sleeve that allows the abrasive cord to slide freely within its lumen, and a pair of handles that fits into the loops at the ends of abrasive cord. The protective sleeve preferable made of a durable, but compressible material. The protective sleeve of the present invention has a longitudinal slit through which the abrasive cord exits when tension is applied to remove a cast applied over it.

The device of this invention is applicable for use in the delivery of healthcare in a less than optimum setting.

It is therefore an objective of the present invention to provide a device for use removal of an applied cast in situations where the use of an electric saw is not feasible.

It is another objective of the present invention to provide a device for removal of an applied cast during emergency medical situations.

It is yet another objective of the present invention to provide a user-friendly device for the removal of an applied cast on an extremity.

### BRIEF DESCRIPTION OF THE FIGURES

The above-mentioned objectives and advantages of the present invention will become more apparent and the invention itself will be best understood by reference to the following description of the present invention taken in conjunction with the accompanying drawings, wherein:

FIG. 1 is a perspective view of the cast removal kit device of the present invention without the handle pieces attached;

FIG. 2 is another perspective view of the cast removal kit device with the handle pieces attached;

FIG. 3 is a perspective view of the cast removal kit device of the present invention with the protective sleeve over the cord.

### DETAILED DESCRIPTION OF THE INVENTION

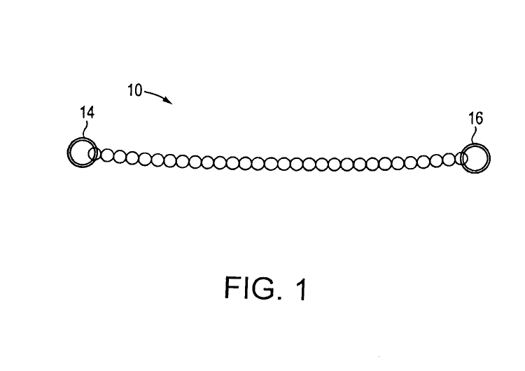

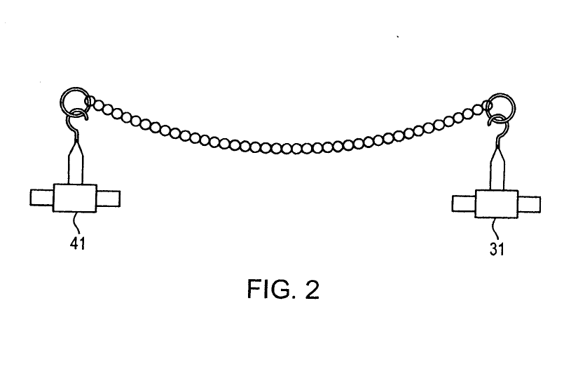

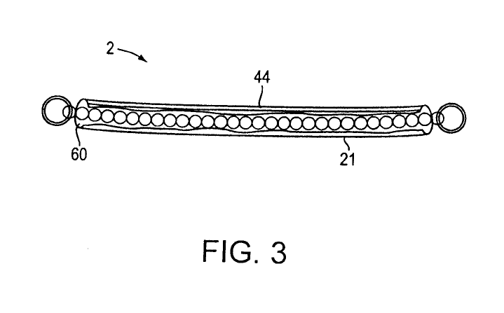

With reference to FIGS. 1, 2 and 3, the device of the current invention is shown. The cast removal kit consists of an elongated abrasive cord 10, having two attached loop 14 and 16—one at each end, preferably of non-abrasive material. The elongated abrasive cord 10 is enclosed in a protective sleeve 21 in a part, or whole segment of its length. The looped ends 14 and 16 at either end of the abrasive cord 10 are left bare for easy access for the attachment of their respective handles 31 and 41. The handles 31 and 41 are shaped in that of a cross, or a T-bar configuration to allow for ease of handling when coupled to the looped ends of the abrasive cord 10. The protective sleeve 21 encases the elongated abrasive cord in a non-tight fitting manner, allowing for a little play within its lumen 60, such that the abrasive cord 10 can slide easily along the axis of the lumen. The elongated protective sleeve is slit along its longitudinal axis 44 such that the encased cord can be pulled through the slit opening during use. The elongated protective sleeve 21 is preferably made of a material that can be cut through with the abrasive cord 10 in the event that the longitudinal slit on the protective sleeve 21 is malpositioned. The elongated protective sleeve 21 is of a flexible and collapsible material such that it will flatten when incorporated in a cast, and not exert unnecessary pressure on the underlying skin. The elongated protective sleeve 21 is preferably of radiolucent material such that it will not obscure the image of a bone subject to a radiographic (X-ray) beam.

The elongated protective sleeve 21 is positioned during use with the slit end pointing upward or, to the sides such that the abrasive cord can be pulled through the slit 44 of the protective sleeve during use to cut an overlying casting material. The abrasive cord 10 is made of one, or more strands of wire, or non-metallic material with comparable cutting properties, the type that is already in use in the stone cutting industry and during bone cutting during hip replacement surgery.

The cast removal kit 2 of the current invention offers a unique method of removing an applied cast on an extremity. The cast removal kit 2 of the current invention also introduces a modification to the way a cast is applied to an extremity. One, or multiple units of the abrasive cord 10 situated in its protective sleeve 21 component of the cast removal kit 2 is positioned above the cotton under-padding and deep to the first layer of an applied cast material. The cast removal kit 2 is then covered with layers of cast material as the extremity is encased in the applied material, with the exception of its looped ends 14 and 16. The positioned cast removal kit 2 is left undisturbed until it is ready to be utilized in the removal of the overlying applied cast. To remove an applied cast, a pair of handles 31 and 41 designed to be easily gripped with the hands, are coupled to the looped ends 14 and 16 of the abrasive cord 10. Tension is then applied to the coupled handles 31 and 41 in an upward direction and maneuvered such that the abrasive cord is moved in a to and fro motion until it makes its way out of the protective sleeve 21 either through the slit opening 44 of the protective slit 21, or by its direct cutting action through the wall of the encasing protective sleeve. The to and fro motion is continued until the abrasive cord cuts through the layers of the overlying cast material. To fully remove an applied cast, at least two units of the cast removal kit is utilized positioned on either side of the cast at the time of cast application. When dealing with a long segment of applied casting material, it may be necessary to apply more than one unit of the cast removal kit 2 on the same side of an applied cast, positioned end-to-end or in series while leaving the looped ends 14 and 16 protruding through the applied cast.

In the preferred form of the current invention, as shown in FIGS. 1, 2 and 3, the combination of the abrasive cord 10, the protective sleeve 21 and the attached handles 31 and 41 make the complete kit 2 for use during the removal of an applied fracture cast.

In another form of the current invention, the attached handles 31 and 41 are pre-configured and incorporated into the looped ends 14 and 16 to be activated during use, such that there is no requirement for separate handles to be coupled to the looped ends of the abrasive cord at the time of use.

In as much as the present invention is subject to many variations, modifications and changes in details, it is intended that all matter described throughout this specification and shown in the accompanying drawings be interpreted as an illustrative depiction and not in a limiting sense.

Accordingly, it is intended that the invention be limited only by the spirit and scope of the hereto appended claims.

The invention claimed is:

1. A cast removal kit comprising:
an elongated cord comprising an abrasive, a first end, and a second end and
an elongated sleeve comprising a longitudinal slit positioned along at least a portion of said elongated sleeve, said sleeve enclosing at least a portion of said elongated abrasive cord;

2. A method of manufacturing a cast removal device, comprising:
combining as a single integral unit an abrasive cord, one or more grips positioned at least one end of said abrasive cord, and an elongated protective sleeve enclosing said abrasive cord, and one or more grip handles.

3. The kit of claim 1, wherein the elongated sleeve comprises a compressible and radiolucent material.

4. A method of removing a cast materials from a body part, comprising:
applying tension to an elongated abrasive cord positioned under said cast material and over the skin of said body part;
moving said elongated cord to and fro;
removing said cast from said body part.

5. A The method of claim 4, wherein moving said elongated cord further comprises moving said elongated cord to and fro.

6. The method of claim 4, further comprising coupling each of said first end and said second end of said elongated abrasive cord to a grip handle.

7. The method of claim 4 further comprising a sleeve positioned over at least a portion of said elongated abrasive end.

8. The method of claim 7 wherein said elongated sleeve comprises an opening along at least a portion of said elongated sleeve.

9. The method of claim 8 wherein said opening comprises a longitudinal slit.

10. The method of claim 8 further comprising pulling said elongated cord through said opening of said elongated sleeve.

11. The kit of claim 1 wherein further comprising a grip positioned at least one of said first end and second end of said elongated cord.

12. The kit of claim 11 wherein said grip comprises a hook.

13. The kit of claim 10 wherein said grip comprises a loop.

14. A method for applying removable cast material to a body part, comprising:
providing an elongated cord comprising an abrasive;
positioning said elongated cord above the skin of said body part;
applying said cast material over said elongated cord.

15. The method of claim 14 wherein said elongated cord is pre-positioned in an elongated sleeve.

16. The method of claim 14 further comprising
enclosing said elongated cord in an elongated sleeve prior to applying said cast material.

17. The method of claim 15 wherein said elongated sleeve comprises an opening positioned along at least a portion of the longitudinal axis of said elongated sleeve.

18. The method of claim 16 wherein said elongated sleeve comprises an opening positioned along at least a portion of the longitudinal axis of said elongated sleeve.

19. The method of claim 17 wherein said opening comprises a slit.

20. The method of claim 18 wherein said opening comprises a slit.
